(19) United States
(12) Patent Application Publication
(10) Pub. No.: US 2023/0233242 A1
(43) Pub. Date: Jul. 27, 2023

(54) CRYOSURGERY SYSTEM FOR SKIN LESION REMOVAL

(71) Applicant: Scholl's Wellness Company LLC, Parsippany, NJ (US)

(72) Inventor: Edmund Farmer, Seymour, CT (US)

(21) Appl. No.: 18/097,807

(22) Filed: Jan. 17, 2023

Related U.S. Application Data
(60) Provisional application No. 63/302,130, filed on Jan. 24, 2022.

(51) Int. Cl. A61B 18/02

(52) U.S. Cl. CPC A61B 18/02; A61B 2018/0225; A61B 17/30

(57) ABSTRACT

Methods, devices, and systems for treatment and removal of skin lesions, such as skin tags, are disclosed herein. In at least one embodiment, a system for treating a skin lesion comprises: a tweezer comprising a treatment region; and an activation station, the activation station configured with a first receiving end and a second receiving end. In at least one embodiment, the activation station receives the tweezer at the first receiving end, and receives a cryogenic agent at the second receiving end. The first receiving end is fluidly coupled to the second receiving end to facilitate delivery of the cryogenic agent to the treatment region of the tweezer.

### CROSS-REFERENCE TO RELATED APPLICATION(S)

This application claims the benefit of priority of U.S. Provisional Patent Application Ser. No. 63/302,130, filed Jan. 24, 2022, the disclosures of which are hereby incorporated herein in their entireties.

### BACKGROUND

Current methods for cryogenic treatment of skin lesions, such as skin tags, utilize a cryogenic agent in the form of a liquid refrigerant contained in a pressurized container. In such methods, an effective amount of the cryogenic agent from the pressurized container is sprayed onto an applicator which is then applied directly to the lesion near the surface of the skin. The application is performed for a period of time sufficient to permit the cryogenic agent to reduce the temperature of the skin lesion tissue to temperatures that freeze the skin, such that permanent, irreversible rupture of cellular membranes of cells of the skin lesion occurs while the cryogenic agent is evaporating. During the next several weeks, the tissue that was frozen dislodges from the surrounding skin.

Problems with current approaches for removing skin tags, in particular, include inefficient loading of the applicator with the cryogenic agent, loss of cryogenic agent during the transfer of the cryogenic agent to the skin tag, and inefficient or incomplete contact between the applicator and the skin tag. Further, current approaches are prone to accidental contact of the cryogenic material with the skin surrounding the skin tag, which can potentially cause injury. Accordingly, there is a need for an improved methods, systems, and devices to treat skin tags.

### SUMMARY

The following presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

One aspect of the present disclosure relates to a method of treating a skin lesion, the method comprising: activating a treatment region of a tweezer with a cryogenic agent; positioning the treatment region of the tweezer on the skin lesion, wherein positioning comprises aligning a pair of absorbent pads provided at the treatment region of the tweezer on opposing sides of the skin lesion; squeezing an articulating end of the tweezer to secure the skin lesion between the pair of treatment heads, the absorbent pads delivering the cryogenic agent to the skin lesion; and maintaining grasp of skin lesion secured between the pair of absorbent pads for a predetermined treatment time.

In at least one embodiment, the cryogenic agent comprises dimethyl ether, liquid nitrogen, or nitrous oxide.

In at least one embodiment, the method further comprises applying a skin shield to an area of treatment comprising the skin lesion, the skin shield enabling isolation and validation of the skin lesion in the area of treatment.

In at least one embodiment, the predetermined treatment time is from about 10 seconds to about 50 seconds.

In at least one embodiment, each step of the method are performed sequentially for a total of one, two, or three or more treatments. In at least one embodiment, subsequent treatments are performed no less than about 10 seconds to about 20 seconds after a prior treatment.

Another aspect of the present disclosure relates to a system for treating a skin lesion, comprising: a tweezer comprising a treatment region; and an activation station, the activation station configured with a first receiving end and a second receiving end, wherein the activation station receives the tweezer at the first receiving end, and wherein the activation station receives a cryogenic agent at the second receiving end, the first receiving end being fluidly coupled to the second receiving end to facilitate delivery of the cryogenic agent to the treatment region of the tweezer.

In at least one embodiment, the activation station comprises: a base portion comprising a loading stage; and a loading portion comprising the first receiving end and the second receiving end.

In at least one embodiment, the base portion and the loading portion are separate components that are mechanically coupled together to form the activation station.

In at least one embodiment the base portion further comprises a shield element and a conical element both protruding from the loading stage, and wherein the loading stage, the conical element, the shield element, and the loading portion collectively form a receiving chamber that facilitates localization of the cryogenic agent onto the treatment region of the tweezer when resting on the loading stage upon injection of the cryogenic agent into the activation station.

In at least one embodiment, the system further comprising a container comprising the cryogenic agent. In at least one embodiment, the cryogenic agent comprises dimethyl ether, liquid nitrogen, or nitrous oxide. In at least one embodiment, the second receiving end comprises one or more protrusions configured to engage with and actuate the container when an actuatable portion of the container is inserted into the second receiving end of the activation station.

Another aspect of the present disclosure relates to a tweezer comprising: an articulating region to actuate the tweezer; and a treatment region. In at least one embodiment, the treatment region comprises: a pair of absorbent pads forming tips of the tweezer and suitable for absorbing a cryogenic agent, each of the absorbent pads having an inward-facing surface and an outward-facing surface; and for each absorbent pad, a pair of treatment shields disposed at opposite edges of the absorbent pad, the treatment shields shaped to allow contact of the inward-facing surface of the absorbent pad and limit contact of the outward-facing surface of the absorbent pad during skin treatment.

In at least one embodiment, the absorbent pads each comprise a foam material.

In at least one embodiment, the treatment shields are formed integrally with the tweezer.

In at least one embodiment, the tweezer further comprises: for each pair of treatment shields, a stabilizer bridge affixed therebetween to substantially cover the outward-facing surface of the respective absorbent pad.

In at least one embodiment, the absorbent pads comprise absorbed cryogenic agent.

Another aspect of the present disclosure relates to a method of activating a tweezer for use in treatment of a skin lesion, the method comprising: inserting a treatment region of the tweezer into a first opening of an activation station, the first opening providing access to a receiving chamber; positioning a container comprising a cryogenic agent in a second opening of the activation station, the second opening configured to deliver the cryogenic agent to the receiving chamber; and actuating the container positioned in the second opening of the activation station, wherein actuation of the container releases the cryogenic agent into the receiving chamber and activates the treatment region of the tweezer.

In at least one embodiment, the cryogenic agent comprises dimethyl ether, liquid nitrogen, or nitrous oxide.

Another aspect of the present disclosure relates to a kit comprising: a tweezer comprising a treatment region; an activation station configured to facilitate delivery of a cryogenic agent to the treatment region of the tweezer when the tweezer is inserted therein; and a container comprising the cryogenic agent.

### BRIEF DESCRIPTION OF THE DRAWINGS

In order to facilitate a fuller understanding of the present disclosure, reference is now made to the accompanying drawings, in which like elements are referenced with like numerals. These drawings should not be construed as limiting the present disclosure, but are intended to be exemplary only.

FIG. 1A illustrates an exemplary activation station in accordance with at least one embodiment of the present invention.

FIG. 1B illustrates an exploded view of an exemplary activation station in accordance with at least one embodiment of the present invention.

FIG. 1C illustrates a cross-sectional view of the exemplary activation station in accordance with at least one embodiment of the present invention.

FIG. 2A illustrates an exemplary tweezer in accordance with at least a first embodiment of the present invention.

FIG. 2B illustrates an exemplary tweezer in accordance with at least a second embodiment of the present invention.

FIG. 3 illustrates an exemplary system comprising a tweezer inserted into a activation station in accordance with at least one embodiment of the present invention.

FIG. 4 is a flow diagram illustrating an exemplary method of treating a skin tag in accordance with at least one embodiment of the present invention.

FIG. 5A illustrates a first step in an exemplary method of treating a skin tag in accordance with at least one embodiment of the present invention.

FIG. 5B illustrates a second step in an exemplary method of treating a skin tag in accordance with at least one embodiment of the present invention.

FIG. 5C illustrates a third step in an exemplary method of treating a skin tag in accordance with at least one embodiment of the present invention.

FIG. 5D illustrates a fourth step in an exemplary method of treating a skin tag in accordance with at least one embodiment of the present invention.

### DETAILED DESCRIPTION

The present disclosure relates generally to methods, systems, and devices for cryogenically treating skin lesions and, more particularly, is directed to an activation station to facilitate delivery of a cryogenic agent onto a treatment end of a tweezer having tips designed to absorb the cryogenic agent. The activation station comprises a first receiving end into which the tips of the tweezer are inserted. The activation station further comprises a second receiving end into which a container of the cryogenic agent is inserted and actuated to release the cryogenic agent onto the tips of the tweezer inserted into the first receiving end.

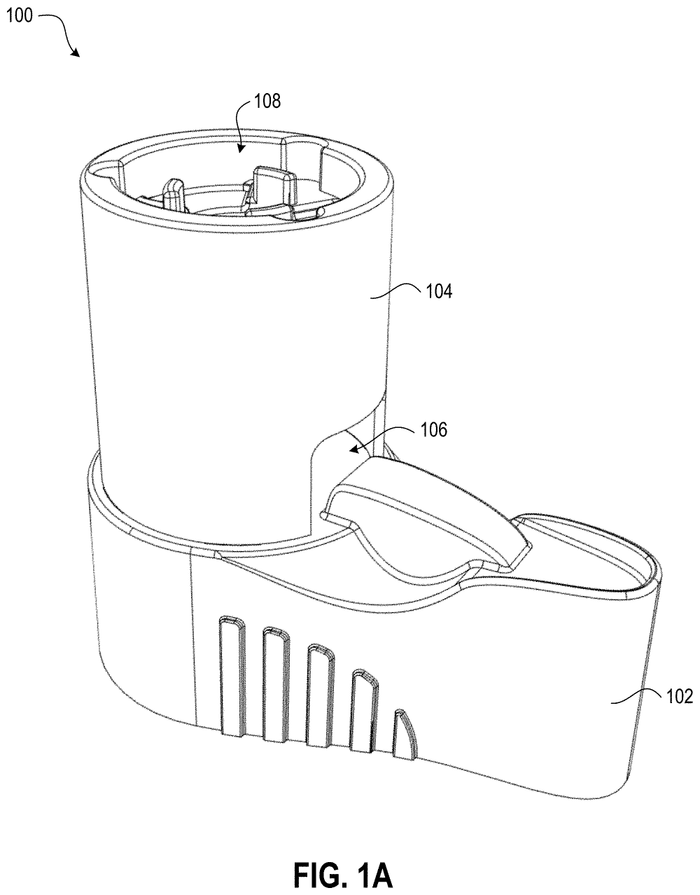

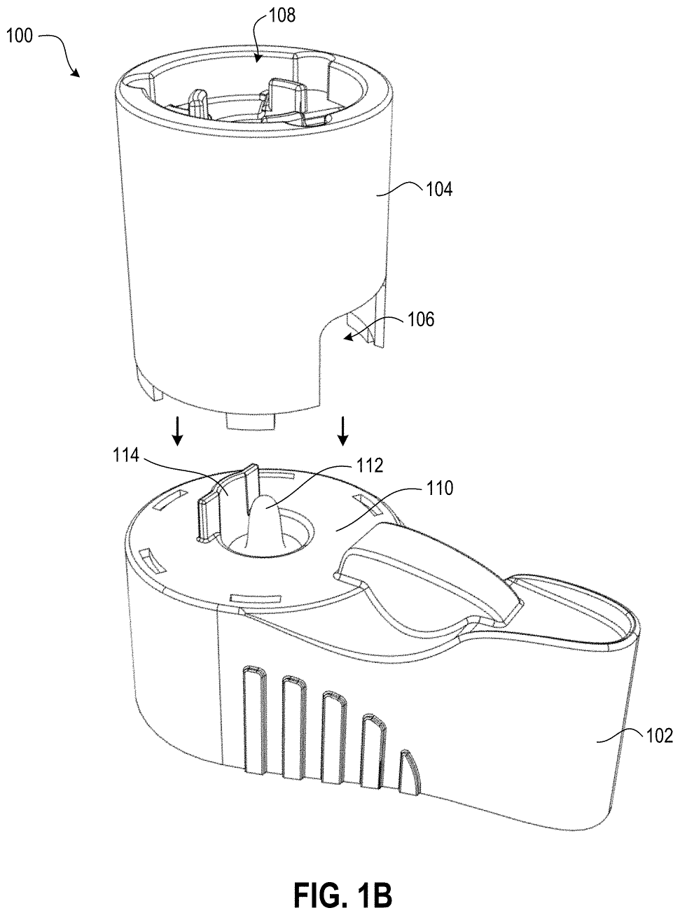

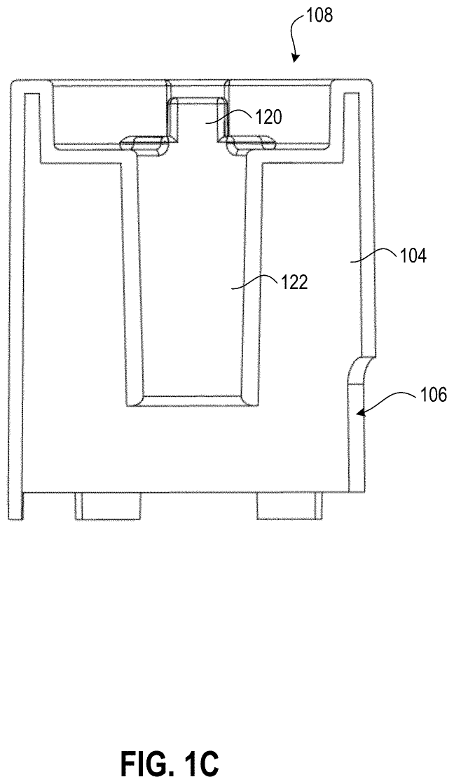

FIGS. 1A-1C illustrate various views of an exemplary activation station 100 and components thereof. The activation station 100 comprises a base portion 102 and a loading portion 104. A tweezer (e.g., tweezers 200 or 250 as discussed below) may be mounted and secured in base portion 102. A container comprising a cryogenic agent may be mounted and secured in loading portion 104. The cryogenic agent may be comprised of dimethyl ether, liquid nitrogen, nitrous oxide, or any other suitable cryogenic agent as would be appreciated by those of ordinary skill in the art. A treatment end of the tweezer may be inserted through an opening 106 formed between base portion 102 and loading portion 104. An actuatable portion of the container may be inserted into an opening 108 of loading portion 104. When the container is actuated, loading portion 104 is constructed to allow for the cryogenic agent to be directed towards base portion 102 so as to fluidly couple the container to the tweezers secured therein. An exemplary container used for storing and delivering a cryogenic agent is described in U.S. Pat. No. 7,604,632 B2, granted on Jul. 20, 2009, the disclosure of which is hereby incorporated by reference herein in its entirety.

In one embodiment, as illustrated in FIG. 1A, activation station 100 is a unitary structure (i.e., not constructed of separate component parts) comprising base portion 102 and loading portion 104. Activation station 100 may be fabricated as a unitary structure, for example, using an additive manufacturing process. In another embodiment, as illustrated in FIG. 1B, activation station 100 may be assembled from separate component parts comprising base portion 102 and loading portion 104. Separate component parts may be, for example, mechanically coupled together (temporarily or permanently) to form activation station 100.

Base portion 102 of activation station 100 comprises a loading stage 110, a conical element 112 and a shield element 114. The treatment end of the tweezer may rest on loading stage 110 when inserted through opening 106. The loading stage, the conical element 112, the shield element 114, and the loading portion collectively form a receiving chamber that facilitates localization of the cryogenic agent onto the treatment end of the tweezer resting on loading stage 110 upon injection of the cryogenic agent into activation station 100. FIG. 1C illustrates a cross section of loading portion 104, which comprises one or more protrusions 120 to engage with and actuate the container when the actuatable portion of the container is inserted into opening 108. A channel 122 fluidly couples the container to the inserted tweezer by guiding the released cryogenic agent toward loading stage 110.

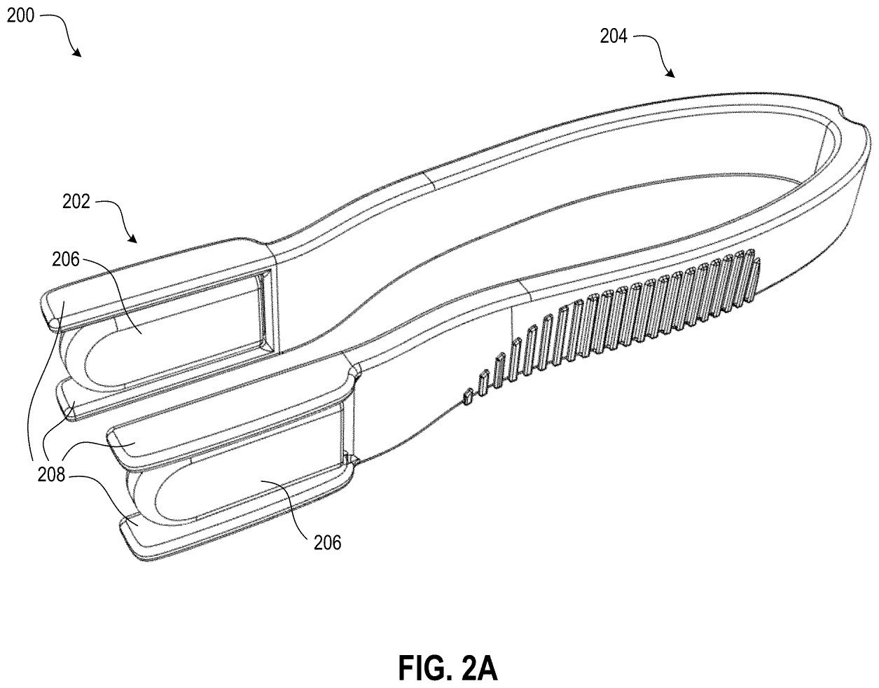

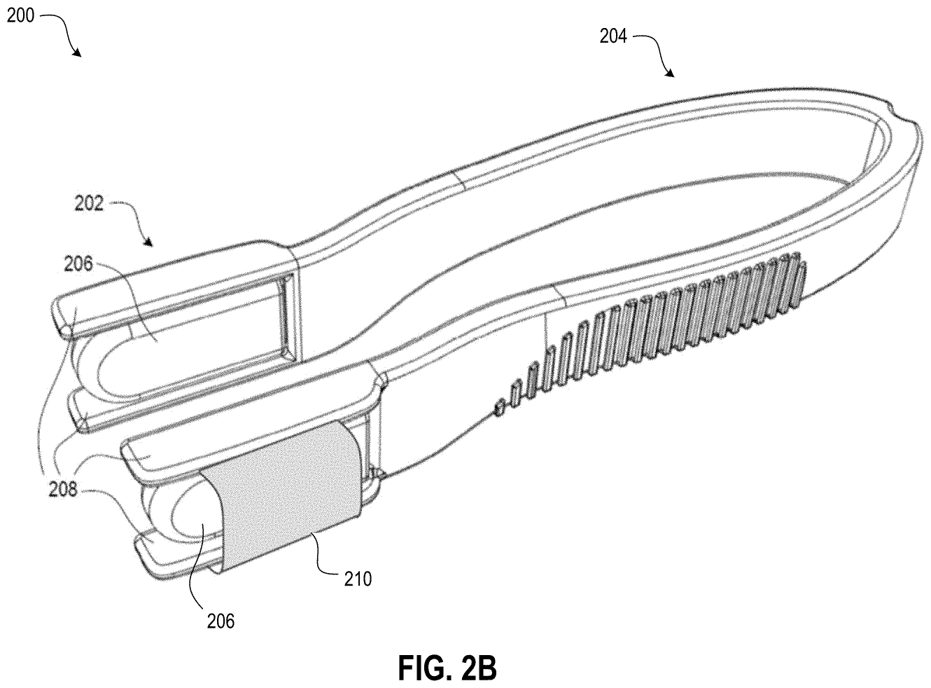

FIG. 2A illustrates the exemplary tweezer 200. Tweezer 200 comprises a treatment region 202 for contacting a protruding skin lesion (such as a skin tag) and an articulating region 204 for actuating tweezer 200. In at least one embodiment, treatment region 202 comprises absorbent pads 206 that form tips of tweezer 200, which may be constructed of a foam material or other suitable thermal insulation material. Each of absorbent pads 206 is disposed between treatment shields 208, such that the treatment shields 208 run along opposite edges of the adsorbent pads while exposing inward-facing and outward-facing surfaces. In at least one embodiment, the treatment shields 208 are shaped to allow contact between the protruding skin lesion with interior sides of absorbent pads 206 while limiting other portions of absorbent pads 206 from contacting the skin during treatment. In another embodiment, as illustrated in FIG. 2B, tweezer 200 may further comprise a stabilizer bridge 210 affixed between upper and lower portions of treatment shields 208. Stabilizer bridge 210 may function to maintain appropriate distance at opposing ends of absorbent pads 206 disposed between the upper and lower portions of treatment shields 208. Additionally, stabilizer bridge 210 may provide added strength to the barrier function offered by treatment shields 208 to prevent improper use or inadvertent contact of absorbent pads 206 against the skin when activated with a cryogenic agent.

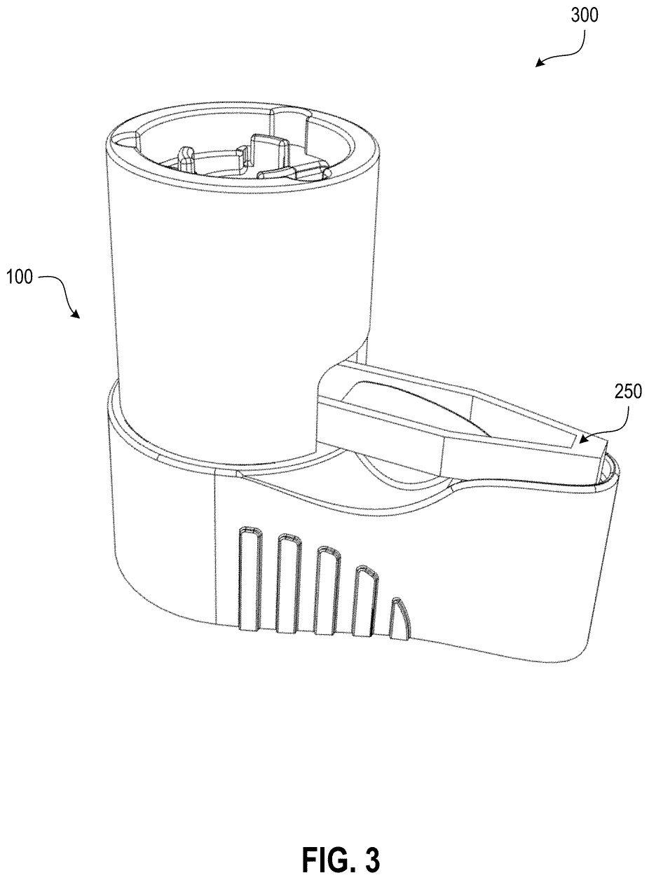

FIG. 3 illustrates an exemplary system 300 comprising a tweezer 250 inserted into activation station 100 in accordance with at least one embodiment. The tweezer 250 may be the same or similar to tweezer 200 illustrated in FIGS. 2A and 2B.

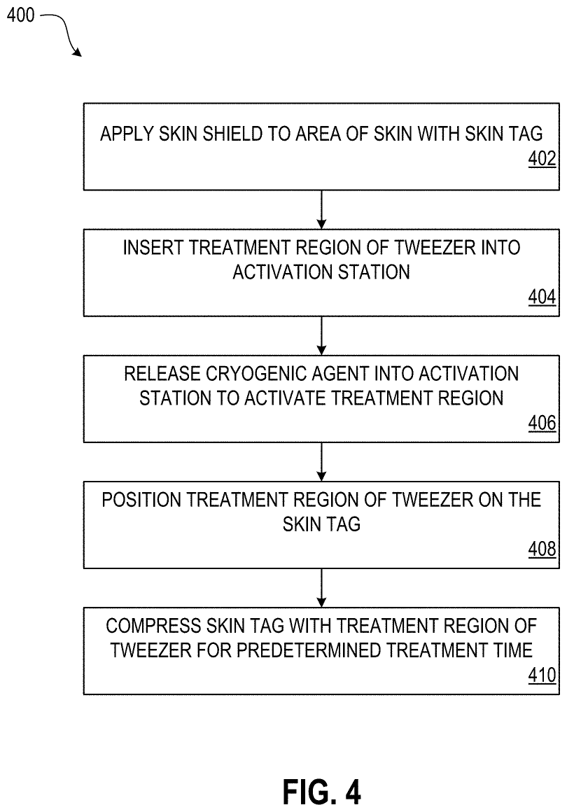

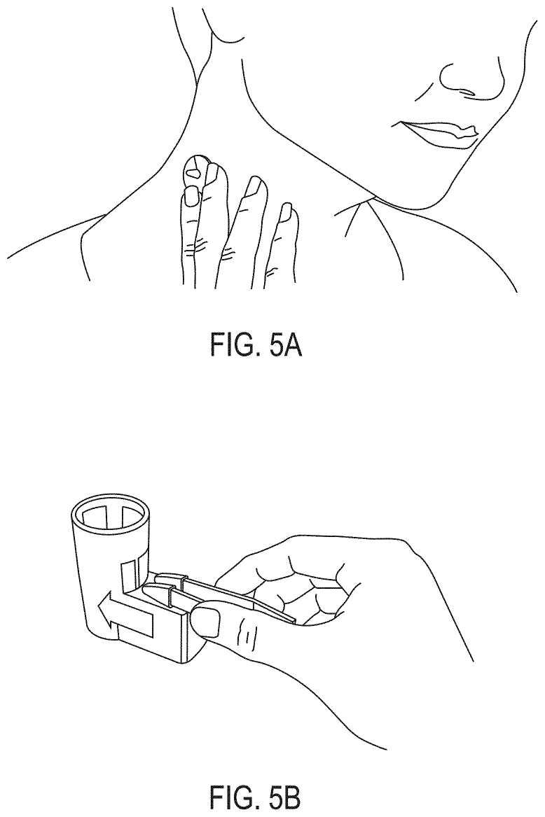

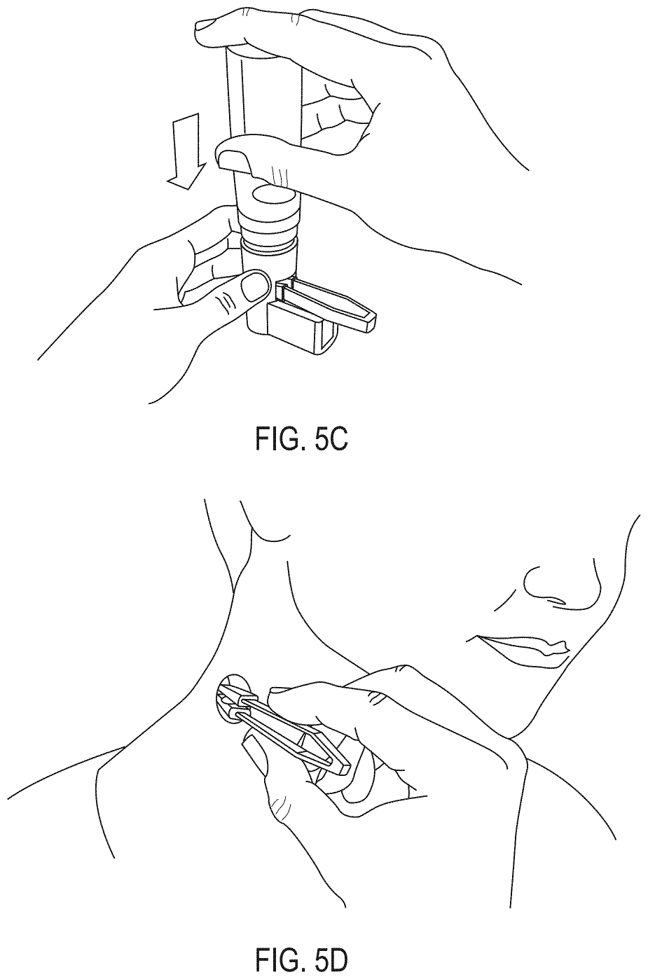

FIG. 4 is a flow diagram illustrating an exemplary method of treating a skin tag in accordance with at least one embodiment. At block 402, a skin shield may be applied to the skin surrounding the skin tag. The skin shield may be a foam disc that covers the surrounding skin while allowing the skin tag to protrude therethrough, as illustrated in FIG. 5A. The skin shield may serve to assist with isolation and validation of the skin tag in the area of treatment. However, use of the skin shield may be omitted from the treatment method. At block 404, a treatment region of a tweezer (e.g., treatment region 202 of tweezer 200) is inserted into a first receiving end of an activation station (e.g., opening 106 of activation station 100), as illustrated in FIG. 5B. At block 406, a container comprising the cryogenic agent is inserted into a second receiving end (e.g., opening 108) of the activation station. When a downward force is applied to the container, the container is actuated to release the cryogenic agent into a receiving chamber in which the treatment region of the tweezer is seated, as illustrated in FIG. 5C. In at least one embodiment, the actuation of the container may be performed for approximately 2-3 seconds. At block 408, the treatment region of the tweezer having received the cryogenic agent is removed and positioned at the skin tag, as illustrated in FIG. 5D. At block 410, the skin tag is compressed with the treatment region of the tweezer for a predetermined treatment time. In at least one embodiment, the treatment time is about 5 seconds, about 10 seconds, about 20 seconds, about 30 seconds, about 40 seconds, about 50 seconds, about 60 seconds, or within any range defined by these time points (e.g., from about 10 seconds to about 50 seconds, from about 30 seconds to about 50 seconds, etc.). As used herein, the term “about” is used to describe and account for small fluctuations. For example, “about” may mean the numeric value may be modified by ±5%. All numeric values are modified by the term “about” whether or not explicitly indicated. Numeric values modified by the term “about” include the specific identified value. For example, “about 5.0” includes 5.0.

After treatment, the skin tag will likely appear to be white and frozen. In at least one embodiment, blocks 402-410 may be repeated for multiple treatments (e.g., 2 or 3 additional treatments) of the protruding skin lesion with 10-20 second intervals between each treatment.

In the foregoing description, numerous details are set forth. It will be apparent, however, to one of ordinary skill in the art having the benefit of this disclosure, that the present disclosure may be practiced without these specific details. For simplicity of explanation, the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter.

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment” or “at least one embodiment” means that a particular feature, structure, or characteristic described in connection with the embodiment is included in at least one embodiment. Thus, the appearances of the phrase “an embodiment” or “at least one embodiment” in various places throughout this specification are not necessarily all referring to the same embodiment.

The present disclosure is not to be limited in scope by the specific embodiments described herein. Indeed, other various embodiments of and modifications to the present disclosure, in addition to those described herein, will be apparent to those of ordinary skill in the art from the description and accompanying drawings. Thus, such other embodiments and modifications are intended to fall within the scope of the present disclosure. Further, while the present disclosure has been described in the context of a particular embodiment in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein, along with the full scope of equivalents to which such claims are entitled.

What is claimed is:

1. A method of treating a skin lesion, comprising:
activating a treatment region of a tweezer with a cryogenic agent;
positioning the treatment region of the tweezer on the skin lesion, wherein positioning comprises aligning a pair of absorbent pads provided at the treatment region of the tweezer on opposing sides of the skin lesion;
squeezing an articulating end of the tweezer to secure the skin lesion between the pair of treatment heads, the absorbent pads delivering the cryogenic agent to the skin lesion; and
maintaining grasp of skin lesion secured between the pair of absorbent pads for a predetermined treatment time.

2. The method of claim 1, wherein the cryogenic agent comprises dimethyl ether, liquid nitrogen, or nitrous oxide.

3. The method of claim 1, further comprising applying a skin shield to an area of treatment comprising the skin lesion, the skin shield enabling isolation and validation of the skin lesion in the area of treatment.

4. The method of claim 1, wherein the predetermined treatment time is from about 10 seconds to about 50 seconds.

5. The method of claim 1, wherein each step of the method are performed sequentially for a total of one, two, or three or more treatments.

6. The method of claim 5, wherein subsequent treatments are performed no less than about 10 seconds to about 20 seconds after a prior treatment.

7. A system for treating a skin lesion, comprising:
a tweezer comprising a treatment region; and
an activation station, the activation station configured with a first receiving end and a second receiving end, wherein the activation station receives the tweezer at the first receiving end, and wherein the activation station receives a cryogenic agent at the second receiving end, the first receiving end being fluidly coupled to the second receiving end to facilitate delivery of the cryogenic agent to the treatment region of the tweezer.

8. The system of claim 7, wherein the activation station comprises:
a base portion comprising a loading stage; and
a loading portion comprising the first receiving end and the second receiving end.

9. The system of claim 8, wherein the base portion and the loading portion are separate components that are mechanically coupled together to form the activation station.

10. The system of claim 8, wherein the base portion further comprises a shield element and a conical element both protruding from the loading stage, and wherein the loading stage, the conical element, the shield element, and the loading portion collectively form a receiving chamber that facilitates localization of the cryogenic agent onto the treatment region of the tweezer when resting on the loading stage upon injection of the cryogenic agent into the activation station.

11. The system of claim 7, further comprising a container comprising the cryogenic agent.

12. The system of claim 11, wherein the cryogenic agent comprises dimethyl ether, liquid nitrogen, or nitrous oxide.

13. The system of claim 11, wherein the second receiving end comprises one or more protrusions configured to engage with and actuate the container when an actuatable portion of the container is inserted into the second receiving end of the activation station.

14. A tweezer comprising:
an articulating region to actuate the tweezer; and
a treatment region, the treatment region comprising:
a pair of absorbent pads forming tips of the tweezer and suitable for absorbing a cryogenic agent, each of the absorbent pads having an inward-facing surface and an outward-facing surface; and
for each absorbent pad, a pair of treatment shields disposed at opposite edges of the absorbent pad, the treatment shields shaped to allow contact of the inward-facing surface of the absorbent pad and limit contact of the outward-facing surface of the absorbent pad during skin treatment.

15. The tweezer of claim 14, wherein the absorbent pads each comprise a foam material.

16. The tweezer of claim 14, wherein the treatment shields are formed integrally with the tweezer.

17. The tweezer of claim 14, further comprising:
for each pair of treatment shields, a stabilizer bridge affixed therebetween to substantially cover the outward-facing surface of the respective absorbent pad.

18. The tweezer of claim 14, wherein the absorbent pads comprise absorbed cryogenic agent.

19. A kit comprising the tweezer of claim 14, and an activation station configured to facilitate delivery of a cryogenic agent to the treatment region of the tweezer when the tweezer is inserted therein.

20. The kit of claim 19, further comprising a container comprising the cryogenic agent.
